(19) United States
(12) Patent Application Publication
(10) Pub. No.: US 2005/0281867 A1
(43) Pub. Date: Dec. 22, 2005

(54) Nitric oxide inducing agents

(76) Inventors: Nighat N. Kahn, Greenlawn, NY (US); Asru K. Sinha, Calcutta (IN)

Correspondence Address: LICATLA & TYRRELL P.C., 66 E. MAIN STREET, MARLTON, NJ, 08053, US

(21) Appl. No.: 11/177,761

(22) Filed: Jul. 8, 2005

Related U.S. Application Data
(63) Continuation-in-part of application No. PCT/US04/01964, filed on Jan. 23, 2004.
(60) Provisional application No. 60/442,439, filed on Jan. 23, 2003.

(51) Int. Cl. A61K 38/28; A61K 33/00; A61K 31/60

(52) U.S. Cl. 424/448; 424/608; 514/3; 514/165

(57) ABSTRACT

The present invention provides a method to promote the production of nitric oxide in cells found in the epidermal layer. The method is effective to prevent and treat a wide range of cancers. The method is also effective for promoting pain relief, controlling diabetes in patients, and treating kidney failure.

### INTRODUCTION

This application claims benefit of priority under 35 U.S.C. §371 to PCT application No. PCT/US2004/01964, filed Jan. 23, 2004, which claims benefit under 35 U.S.C. §119 to U.S. Provisional Patent Application Serial No. 60/442,439, filed on Jan. 23, 2003, whose contents are incorporated herein by reference in their entireties.

### BACKGROUND OF THE INVENTION

Nitric oxide is a chemical that has been implicated in many processes in the body, including regulation of blood pressure, defense against infection, function of the platelets and transmission of some types of nerve impulses. Nitric oxide has been implicated in neurotoxicity associated with stroke and neurodegenerative diseases, neuronal regulation of smooth muscle including peristalsis, and penile erections. Nitric oxide has been proposed to be a messenger molecule for its diversified effects in various physiologic and pathologic events (Ignarro (1990) Ann. Rev. Pharmocol. Toxicol. 30:535-560). Unlike typical neurotransmitters, nitric oxide is not stored in synaptic vesicle and does not act on membrane receptors.

Incubation of various tissues including heart, liver, kidney, muscle and intestine from mice and erythrocytes or their membrane fractions from humans with physiologic concentrations of insulin has resulted in activation of a membrane-bound nitric oxide synthase (NOS), which is distinct from the NOS which is designated the inducible form of NOS (iNOS). Activation of NOS and synthesis of nitric oxide were stimulated by the binding of insulin to specific receptors on the cell surface (Kahn, et al. (2000) IUBMB Life 49:441-450). It was further demonstrated that a membrane-bound form of NOS of human erythrocytes could be activated by insulin (Bhattacharya, et al. (2001) Arch. Physiol. Biochem. 1009:(5)441-449). Insulin has been established to have an essential role in carbohydrate metabolism. Currently, insulin treatment is used for blood sugar-related conditions.

The present invention provides a method to promote the production of nitric oxide in cells found in the epidermal layer. The method is effective to prevent and treat a wide range of cancers. The method is also effective to promote pain relief in patients, to control blood sugar in Type I and Type II diabetics and treat kidney failure.

### SUMMARY OF THE INVENTION

The present invention provides a method for preventing and treating cancer without causing significant side effects to a human patient in need thereof, by topically administering an agent which modulates the production of nitric oxide by cells found in the epidermal tissue layer of a patient.

The present invention also provides a method for relieving pain in a patient without causing significant side effects by topically administering an agent which modulates the production of nitric oxide by red blood cells.

The present invention further provides a method for decreasing the systemic side effects of cancer treatment in a patient by topically administering an agent which modulates the production of nitric oxide by cells found in the epidermal tissue layer of a patient.

The present invention still further provides a method for controlling or preventing diabetes in a patient by topically administering an agent which modulates the production of nitric oxide by cells found in the epidermal tissue layer of a patient.

The present invention also provides a method for treating kidney failure in a human patient in need thereof, by topically administering an agent which modulates the production of nitric oxide by cells of a patient so that kidney function is restored.

In particular embodiments of the instant methods, aspirin is co-administered with the agent which modulates the production of nitric oxide by cells of the patient.

### DETAILED DESCRIPTION OF THE INVENTION

Nitric Oxide (NO) has been reported to possess a wide range of antineoplastic properties. However, until the present invention, the identity of the physiologic stimulator of NO synthesis remained obscure. The present invention demonstrates the existence of an insulin-activated nitric oxide synthase (IANOS) in various cell membranes. In cancerous tumors an antibody is produced that blocks insulin-activated nitric oxide synthase (IANOS). The enzymatic activity of the erythrocyte membranes from patients with different types of neoplastic conditions is markedly inhibited due to the presence of an antibody which results in the diminished synthesis of NO in the patient's system. The present invention identifies agents which are able to neutralize the antibody in vitro from both biologic sources and from non-biologic sources. These agents act through in situ generation of nitric oxide which results in not only amplification of the enzymatic activity but also the neutralization of the antibody in vivo.

It is believed that neoplastic cells elicit the aid of an antibody in the system capable of blocking the production of nitric oxide through the activation of IANOS by insulin. Unlike normal cells, cancer cells do not produce nitric oxide when treated with insulin. Further, cancer cells do not need insulin for the stimulation of carbohydrate metabolism. Nitric oxide only stimulates carbohydrate metabolism in normal cells. However, nitric oxide acts as a potent tumoricide in cancerous cells. The antibody against IANOS plays a crucial role in the pathophysiology of cancer by blocking IANOS. The antibody against IANOS is the light chain part of IgG. This antibody occurs in both humans and animals with neoplastic diseases and cancers.

The present invention provides a method for treating cancer without causing significant side effects to a human patient in need thereof, by providing an agent which modulates the production of nitric oxide by cells found in the epidermal layer in a patient. The agent is prepared in a formulation suitable for topical application to the patient (e.g., via ointment, cream, lotion, paste, gel, spray, aerosol, oil, patch or other pharmaceutical formulation). Topical formulation and methods for producing the same are well-known in the art. See, e.g., Remington: The Science and Practice of Pharmacy, Alfonso R. Gennaro, editor, 20th ed. Lippingcott Williams & Wilkins: Philadelphia, Pa., 2000. In one embodiment, the agent which modulates production of nitric oxide is applied to the skin of the patient via a dermal patch. The dermal patch may contain an adhesive backing for attachment to the skin of the patient. The dermal patch may further contain a backing material which renders the patch impermeable to oils and water. The formulation should be applied near the tumor site in the patient. In particular embodiments, the agent which modulates the production of nitric oxide synthase in cells found in the epidermal tissue layer of a patient is insulin or sodium nitroprusside (Na₂Fe[(CN)5NO]) in water.

Importantly, the neutralization of IANOS antibody is completed only by dermal application of agents which modulate the production of nitric oxide in cells of a patient. The agents themselves do not need to enter into circulation in the patient. Dermal application allows the agents to penetrate the skin of the patient and activate NO synthesis in red blood cells. In cancer patients, application of the agents in manners other than topical or transdermal application have not been found to be effective.

The IANOS which is present in various cell membranes is actually an insulin receptor. In cancer cells, IANOS is blocked by an antibody (light chain of IgG). However, in the skin cell membranes the antibody does not block IANOS because the antibody is not present in the skin surface. Like insulin, nitric oxide activates IANOS, but unlike insulin, the activation of IANOS by nitric oxide is initiated even in the presence of the antibody. Hence, nitric oxide can subsequently diffuse into the circulation without the modulating agent entering into the circulation. However, the diffusion of nitric oxide into the circulation in turn activates erythrocyte membrane IANOS. This amplification of IANOS activity of the erythrocyte membrane further results in increased plasma nitric oxide levels in the patient.

When a dermal patch containing an agent of the instant invention is applied to the patient's skin, synthesis of nitric oxide in cells of the epidermal layer is induced by the agent. The patch is not intended to accomplish transdermal delivery of the agent into the circulation of the patient. Rather, nitric oxide production is achieved. Increased nitric oxide activates enzymes which block the anti-IANOS antibody. Once the anti-IANOS antibody is blocked, the nitric oxide begins to act as a tumoricide in cancer cells.

An agent which modulates the production of nitric oxide by red blood cells, is any agent which induces systemic production of nitric oxide, and which counteracts the antibody to IANOS. The induction of nitric oxide reactivates antibody inhibited IANOS in cancer patients. In particular embodiments, agents which modulate the production of nitric oxide by cells found in the epidermal layer are insulin and Na₂Fe[(CN)5NO] in water. In certain embodiments, the insulin is of bovine pancreatic origin.

The duration of treatment varies with each patient. However, a typical range of topical application is between about 30 and 45 days. After 30 days of treatment via a dermal patch, some patients continued to neutralize the anti-IANOS antibody despite a discontinuation of patch application. This continued neutralization indicated that nitric oxide production in patients was restored. Other patients still required continued topical application to maintain nitric oxide production.

Moreover, it was observed that a few patients who received treatment with an agent of the instant invention began to develop resistance due to down-regulation of the insulin receptor. This effect was reversed by co-administering low dose aspirin (15 mg) with the agent. Upon co-administration, the production of nitric oxide returned to normal within 30 minutes of treatment. While the instant agents work through the insulin receptor, aspirin appeared to be insulin-independent and was nitric oxide mediated. Accordingly, one embodiment of the instant methods is the co-administration, i.e., simultaneous or sequential, of low dose aspirin (e.g., ranging from 5-100 mg) with an agent of the instant invention.

Treatment with modulating agents of the present invention has the benefit of being non-toxic and non-invasive when compared with chemotherapy and surgical options. The only side effect observed was that 1-2 percent of patients developed hypoglycemia. As shown in Table 1, both compositions, namely, insulin and Na₂Fe[(CN)5NO] in water, were found to be effective against a wide variety of cancers. Insulin was particularly effective in non-Hodgkin's lymphoma, brain cancer, breast cancer with mastectomy, and lung cancer (non-small cell). Whereas the composition containing Na₂Fe[(CN)5NO] in water was particularly effective in lung cancer, breast cancer without mastectomy, esophagus, liver cancer, gall bladder cancer, colon cancer, rectum cancer, acute lymphocytic leukemia, acute myeloid leukemia, multiple myeloma, uterine cancer, cervical cancer, Hodgkin's lymphoma, renal cell carcinoma, ovarian cancer, prostrate cancer, tongue cancer, pyriform fossa, and mandible cancer. Both agents were equally effective against pancreatic cancer and bone cancer.

In addition, the increase in systemic NO levels associated with insulin and Na₂Fe[(CN)5NO] in water administration resulted in the increase of both maspin and alpha interferon-a in malignant breast cancer tissue and in non-malignant neutrophils in breast cancer. Expression of maspin, a serine protease inhibitor (serpin) known to be a potent anticancer protein, is severely impaired in breast cancer tissue. Increases in nitric oxide levels effectively restored the production of maspin in the breast cancer tissues of subjects disclosed herein. Similarly, production of alpha interferon-a, a widely used anticancer cytokine noted for its antiproliferative and anticancer property, is severely impaired in both malignant breast cancer tissue and non-malignant neutrophils in breast cancer. As with maspin, increases in nitric oxide levels increased production of alpha-interferon to normal ranges. By administering an agent of the instant invention, the auto immunity that is T-cell induced is improved through the neutrophils.

Treatment methods using agents which modulate the production of nitric oxide is non-invasive and does not produce any discernable side effects, such as toxicity, that are commonly experienced by patients undergoing surgery or receiving chemotherapy or radiotherapy. In terms of a therapeutic approach, the methods of treatment using agents which modulate the production of nitric oxide are more effective than other existing treatments.

TABLE 1
Effects of Application of Antineoplastin for 45 Days on Several Hematological Tests in Patients with
Different Cancer and on the Survival of These Patients Due to Continued Use of the Agent

| | | Hematological | | | | |
|---|---|---|---|---|---|---|
| | | Test | | | | |
| | | (Improvement | | | Tabulated | |
| | | in Hb, TC, DC, | | | Tau | |
| Types of | Types of | or any other | Improvement | | (τ) at | Survival % |
| Cancer | Patient | test as | in | Obsvd. | 5% | (after |
| (Diagnosis) | (n) | indicated) (n) | LFT (n) | (T) | Level | 2 yrs) |
| Lungs Non | R | 102 | 98 | | | 59 |
| Small Cell | (152) | | | | | |
| Carcinoma | | | | | | |
| (Age 45-72) | | | | | | |
| M F | I | | | 0.0129 | −1.645 | |
| (145) (65) | | | | | | |
| Diagnosed by | NR | 3 | 5 | | | 5 |
| histopathology | (58) | | | | | |
| after | | | | | | |
| bronchoscopy, | | | | | | |
| X-ray | | | | | | |
| Lungs Small | R | 80 | 79 | | | 59 |
| Cell Carcinoma | (95) | | | | | |
| (Age 30-65) | | | | | | |
| M F | II | | | 0.0150 | −1.645 | |
| (65) (60) | | | | | | |
| Diagnosed by | NR | 2 | 3 | | | 2 |
| histopathology | (30) | | | | | |
| bronchoscopy, | | | | | | |
| X-ray | | | | | | |
| Breast Cancer | R | 470 | 480 | | | 65 |
| (without | (520) | | | | | |
| mastectomy) | | | | | | |
| (Age 25-55) | | | | | | |
| F (600) | II | | | 0.0128 | −1.645 | |
| Diagnosed by | NR | 16 | 7 | | | 22 |
| mammography | (80) | | | | | |
| and biopsy as | | | | | | |
| infiltrating | | | | | | |
| ductal cells | | | | | | |
| or invasive | | | | | | |
| lobular with | | | | | | |
| axillary lymph | | | | | | |
| node | | | | | | |
| metastasis | | | | | | |
| AcuteLymphoblastic | R | 103 improved | | | | 70 |
| Leukemia | (125) | to normal | | | | |
| (Age 4-70) | | condition, | | | | |
| | | blast cells | | | | |
| | | not found | | | | |
| M F | II | | ND | 0.0357 | −1.645 | |
| (105) (45) | | | | | | |
| Diagnosed by | NR | 2 | | | | 1 |
| blood picture | (25) | | | | | |
| and bone | | | | | | |
| marrow test | | | | | | |
| Acyte Myeloid | R | 50 patients | | | | 67 |
| Leukemia | (65) | had no | | | | |
| (Age 5-75) | | immature | | | | |
| | | lymphoid | | | | |
| | | cells, no | | | | |
| | | nucleated RBC | | | | |
| | | & normal Hb, | | | | |
| | | TC, DC | | | | |
| M F | II | | ND | 0.0404 | −1.645 | |
| (45) (35) | | | | | | |
| Diagnosed by | NR | 0 | | | | 1 |
| bone marrow | (15) | | | | | |
| tests and | | | | | | |
| blood picture | | | | | | |
| Multiple | R | 38 had Bence | | | | 72 |
| Myeloma | (45) | Jones protein | | | | |
| (Age 30-60) | | negative; A2 | | | | |
| | | Macroglobulin | | | | |
| | | within normal | | | | |
| | | limits | | | | |
| M F | II | | ND | 0.0694 | −1.645 | |
| (45) (15) | | | | | | |
| Diagnosed by | (NR) | 2 | | | | 1 |
| biopsy and | (35) | | | | | |
| blood electrophoresis | | | | | | |
| Uterus | R | 600 | 600 | | | 68 |
| (Age 35-60) | (815) | | | | | |
| F (1020) | II | | | 0.0121 | −1.645 | |
| Diagnosed by | NR | 14 | 3 | | | 8 |
| biopsy, FNAC, | (205) | | | | | |
| USG | | | | | | |
| Cervix | R | 625 | 510 | | | 67 |
| (Age 35-60) | (761) | | | | | |
| F (998) | II | | | 0.0114 | −1.645 | |
| Diagnosed by | NR | 12 | 10 | | | 9 |
| biopsy, FNAC, | (237) | | | | | |
| USG | | | | | | |
| Prostate | R | 67 (most | 52 | | | 68 |
| (Age 35-75) | (78) | patients had | | | | |
| | | initial level | | | | |
| | | of PSA | | | | |
| | | (>100 ng/ml), | | | | |
| | | it was | | | | |
| | | normalized to | | | | |
| | | 4 ng/ml after | | | | |
| | | one month | | | | |
| M (95) | II | | | 0.0396 | −1.645 | |
| Diagnosed by | NR | 0 | | | | 1 |
| serum PSA, | (17) | | | | | |
| acid | | | | | | |
| phosphatase | | | | | | |
| test, USG and | | | | | | |
| biopsy | | | | | | |
| Glioma | R | 79 | | | | 60 |
| (Age 30-72) | (82) | | | | | |
| M F | I | | ND | 0.0099 | −1.645 | |
| (65) (37) | | | | | | |
| Diagnosed by | NR | 1 | | | | 1-2 |
| CT Scan | (20) | | | | | |

The present invention further provides a method for relieving pain in a patient without causing significant side effects by providing an agent which modulates the production of nitric oxide by cells found in the epidermal layer in a patient, and topically delivering the agent to said patient. In particular embodiments, topical administration of the agent is completed via a dermal patch applied to the skin of said patient; however, any other topical administration mode may be used such as ointments, creams, lotions, and the like.

The present invention further provides a useful method for preventing cancer in a human patient comprising topically administering an agent which modulates the production of nitric oxide by cells found in the epidermal layer of a patient. As shown in Table 2, a study of 590 patients with simple radial mastectomy were topically administered an agent which modulates the production of nitric oxide by cells found in the epidermal layer in the patient after the removal of the cancerous tumor tissue. 480 of the patients favorably responded to the treatment, and of the 480 patients who responded to the treatment, 40 percent of the patients survived for at least two years as opposed to only seven percent of the patients who did not respond to the treatment. Moreover, occurrence of cancer metastasis in patients administered an agent of the instant invention was very low (<0.01%). These results strongly indicate that treatment with an agent which modulates the production of nitric oxide by cells found in the epidermal layer actually prevents cancer from progressing in patients (e.g., via cancer recurrence or metastasis) and is therefore useful for providing prolonged disease-free survival. As used herein, prolonged disease-free survival is intended to mean disease-free survival (i.e., no cancer recurrence) for generally more than 2 years after treatment.

The present invention further provides a method for improving or reducing the systemic side effects associated with cancer in a patient comprising providing an agent which modulates the production of nitric oxide by cells found in the epidermal layer by topically applying the agent to said patient. As shown in Table 2, the systemic side effects associated with cancer in a patient include, but are not limited to: pain and discomfort, abdominal distention, iron lymphedema, irregular hemoglobin count, irregular serum PSA, hematuria, neurological problems with vision and memory, swelling, dysphagia, irregular white blood cell count, appetite loss, nausea, increased oedema, impaired electrolytic balance, irregular amounts of protein found in patients blood, irregular amounts of A2 macro-globulin found in patients blood, and irregular swelling of lymph nodes

The present invention also provides a method for controlling or preventing diabetes from occurring in patients having or at risk of acquiring diabetes (e.g., overweight or pregnant) by topically administering an agent which modulates the production of nitric oxide by cells found in the epidermal layer in a patient. In particular embodiments, the agent which modulates the production of nitric oxide synthase by the red blood cells of a patient is insulin or Na₂Fe[(CN)5NO] in water. In further embodiments, the patient is a type I diabetic, a type II diabetic or a cancer patient.

TABLE 2
Effect of Agents of the Instant Invention on Various Types of Cancer

| | | | | | | | Condition | |
|---|---|---|---|---|---|---|---|---|
| | | | | | | | Observed | |
| | Condition | | | | Hb | | After | |
| | at | | Reduced | Appetite | Stable | | Application | % |
| Cancer Type | Presentation | | Pain | Increase | or Improvement | LFT | of agent | Survival |
| Lungs Non | Severe | R | 102 | 110 | 102 | 98 | Reduced | 59% |
| Small Cell | chest | | | | | | pleural | |
| Carcinoma | pain with | | | | | | effusion, | |
| (Age 45-72) | loss of | | | | | | patchopacities, | |
| M F | appetite, | | | | | | supraclavicular | |
| (135) (65) | cough, | | | | | | lymph node | |
| Diagnosed | fever, | NR | 6 | 5 | 3 | 4 | metastasis, | 5% |
| by histopathology | respiratory | | | | | | cough and | |
| after | distress, | | | | | | respiratory | |
| bronchoscopy, | cachexia, | | | | | | distress. | |
| X-ray | anorexia, | | | | | | Improvement | |
| | parenchymal | | | | | | of pain, | |
| | lesion, | | | | | | appetite, | |
| | haeoptysis. | | | | | | weight. | |
| Lungs Small | Severe | R | 110 | 115 | 80 | 79 | Reduced | 59% |
| Cell | chest and | | | | | | pleural | |
| Carcinoma | back pain | | | | | | thickening | |
| (Age 30-65) | with loss | | | | | | Infiltration | |
| M F | of | | | | | | of | |
| (90) (60) | appetite, | | | | | | left | |
| Diagnosed | mulemphysematous | NR | 2 | 1 | 3 | 3 | lingular | 2% |
| by histopathology | bullae in | | | | | | lobe, lower | |
| after | cases, | | | | | | lobe of | |
| bronchoscopy | cough, | | | | | | bronchii | |
| X-ray | severe | | | | | | pain with | |
| | pleffusion, | | | | | | cough, | |
| | respiratory | | | | | | problems, | |
| | distress, | | | | | | hemoptysis | |
| | haemoptysis, | | | | | | totally | |
| | cachexia, | | | | | | reduced (in | |
| | anorexia. | | | | | | some | |
| | | | | | | | cases). | |
| Larynx and | Hoarseness | R | 100 | 130 | 82 | 79 | Specimen | 69% |
| NasophaRynx | of | | | | | | from | |
| (Age 35-55) | voice, | | | | | | nasopharyngeal | |
| M F | inability | | | | | | larynx | |
| (120) (38) | to speak | | | | | | mass showed | |
| Diagnosed | (some | NR | 2 | 1 | 2 | 2 | extensive | 1% |
| by punch | cases), | | | | | | fibrosis. | |
| biopsy | lymph | | | | | | Few | |
| | adenopathy, | | | | | | malignant | |
| | excess | | | | | | cells, | |
| | saliva, | | | | | | induction | |
| | swelling | | | | | | of | |
| | of neck, | | | | | | apoptosis | |
| | cachexia, | | | | | | normalized, | |
| | anorexia, | | | | | | decreased | |
| | pain. | | | | | | lymphadenoathy, | |
| | | | | | | | swelling | |
| | | | | | | | totally | |
| | | | | | | | reduced | |
| | | | | | | | with | |
| | | | | | | | increased | |
| | | | | | | | food | |
| | | | | | | | intake. | |
| Breast | Acute | R | 490 | 502 | 470 | 480 | Decrease in | 65% |
| Cancer | pain in | | | | | | regional or | |
| (without | shoulders | | | | | | axillary | |
| mastectomy) | lack of | | | | | | lymph | |
| (Age 25-55) | appetite, | | | | | | adenopathy. | |
| F (600) | open | | | | | | The breast | |
| Diagnosed | wound in | NR | 3 | 2 | 3 | 3 | tumor first | 22% |
| by xerogram | the | | | | | | showed | |
| mammograms | breast, | | | | | | necrosis, | |
| and biopsy | with pain | | | | | | bleeding | |
| as | in right/ | | | | | | with | |
| infiltrating | left | | | | | | mucous, | |
| ductal | hand, | | | | | | then the | |
| cells or | cachexia | | | | | | wound | |
| invasive | (in some | | | | | | started | |
| lobular | cases). | | | | | | healing | |
| with | | | | | | | | |
| axillary | | | | | | | | |
| lymph node | | | | | | | | |
| metastasis | | | | | | | | |
| Breast | Acute | R | 480 | 520 | 425 | 460 | Lymphadenopathy/ | 40% |
| Cancer | pain in | | | | | | lymphatic | |
| (with | either | | | | | | obstruction | |
| simple/ | left/ | | | | | | decreased, | |
| radical | right | | | | | | considerable | |
| mastectomy) | hand | | | | | | reduction | |
| (Age 32-65) | depending | | | | | | of swelling | |
| F (590) | on the | | | | | | of hands | |
| Patients | positions | NR | 4 | 3 | 5 | 4 | and | 7% |
| had | of | | | | | | cachexia. | |
| axillary | mastectomy. | | | | | | | |
| lymphnode | Lack of | | | | | | | |
| metastasis. | appetite, | | | | | | | |
| In some | anorexia, | | | | | | | |
| cases | constipation. | | | | | | | |
| sternum | | | | | | | | |
| mediasturnum | | | | | | | | |
| metastasis | | | | | | | | |
| reported. | | | | | | | | |
| Oesophagus | Severe | R | 580 | 570 | 570 | 540 | Reduced | 80% |
| (Age 35-50) | pain | | | | | | problems in | |
| M F | problem | | | | | | degalutition, | |
| (450) (159) | in | | | | | | dysphagis, | |
| Diagnosed | deglutition. | NR | 1 | 1 | 1 | 2 | improvement | 3% |
| by barium | Most | | | | | | in | |
| swallow | patients | | | | | | cachexia, | |
| X-ray and | were fed | | | | | | patients | |
| GI | through | | | | | | could eat | |
| endoscopy | ryle's | | | | | | solid food | |
| | tube, | | | | | | in 60% | |
| | dysphagia. | | | | | | cases, no | |
| | | | | | | | sign of | |
| | | | | | | | malignancy. | |
| Stomach | Patients | R | 210 | 196 | 184 | 185 | | 45% |
| (Age 28-60) | having | | | | | | | |
| M F | acute | | | | | | | |
| (155) (95) | abdominal | | | | | | | |
| Diagnosed | pain, | NR | 2 | 2 | 3 | 2 | | 5% |
| by biopsy, | nausea, | | | | | | | |
| endoscopy, | cachexia, | | | | | | | |
| USG | anorexia, | | | | | | | |
| | abdominal | | | | | | | |
| | distention. | | | | | | | |
| Liver | Patients | R | 220 | 230 | 135 | 128 | Nausea | 38% |
| (Age 28-60) | reported | | | | | | decreased, | |
| M F | severe | | | | | | reduction | |
| (175) (100) | nausea, | | | | | | in SGOT, | |
| Diagnosed | lack of | NR | 2 | 2 | 3 | 3 | SGPT | 4% |
| by USG, CT, | appetite, | | | | | | values, | |
| biopsy | peritoneal | | | | | | peritoneal | |
| | fluid | | | | | | accumulation | |
| | accumulation, | | | | | | sharply | |
| | abnormal | | | | | | reduced. | |
| | blood | | | | | | Improved | |
| | counts, | | | | | | anemic | |
| | acute | | | | | | conditions | |
| | abdominal | | | | | | and | |
| | pain. | | | | | | cachexia. | |
| Pancreas | Severe | R | 76 | 65 | 81 | 53 | CBD | 38% |
| (Age 28-65) | jaundice, | | | | | | obstruction | |
| M F | loss of | | | | | | was totally | |
| (98) (25) | appetite, | | | | | | normalized | |
| Diagnosed | extreme | NR | 2 | 2 | 2 | 3 | and | 1% |
| by biopsy, | cachexia, | | | | | | hypodense | |
| USG. | abdominal | | | | | | areas could | |
| | distention, | | | | | | not be | |
| | in | | | | | | located. In | |
| | some | | | | | | cases, high | |
| | cases. | | | | | | bilirubin | |
| | | | | | | | counts and | |
| | | | | | | | abnormal | |
| | | | | | | | LFT results | |
| | | | | | | | antineoplastin | |
| | | | | | | | plastin was | |
| | | | | | | | 60% | |
| | | | | | | | effective. | |
| | | | | | | | In | |
| | | | | | | | contrast, | |
| | | | | | | | patients | |
| | | | | | | | with severe | |
| | | | | | | | metastasis | |
| | | | | | | | and | |
| | | | | | | | cachexia | |
| | | | | | | | responded | |
| | | | | | | | to antineoplastin. | |
| Gall | In some | R | 120 | 135 | 98 | 56 | Obstructive | 39% |
| Bladder | cases | | | | | | jaundiced | |
| (age 30-65) | there | | | | | | patients | |
| M F | were CBD | | | | | | lowered | |
| (100) (50) | obstruction, | | | | | | bilirubin | |
| Diagnosed | severe | NR | 1 | 1 | 1 | 3 | counts | 1% |
| by | jaundice, | | | | | | improved | |
| biopsy/CT/ | extreme | | | | | | cachexia | |
| USG. | cachexia | | | | | | and | |
| | and | | | | | | anorexia. | |
| | anorexia. | | | | | | USG results | |
| | | | | | | | showed a | |
| | | | | | | | decrease of | |
| | | | | | | | calculus/ | |
| | | | | | | | SOL in the | |
| | | | | | | | gall | |
| | | | | | | | bladder. No | |
| | | | | | | | signs of | |
| | | | | | | | previous | |
| | | | | | | | metastasis | |
| | | | | | | | could be | |
| | | | | | | | located. | |
| | | | | | | | Patients | |
| | | | | | | | with | |
| | | | | | | | previous | |
| | | | | | | | cholecysctectomy | |
| | | | | | | | and | |
| | | | | | | | cholelithiasis | |
| | | | | | | | showed | |
| | | | | | | | significant | |
| | | | | | | | improvement. | |
| Colon | Acute | R | 192 | 186 | 175 | 184 | Anorectal | 69% |
| (Age 35-71) | electrolytic | | | | | | bleeding | |
| M F | disbalance. | | | | | | and pain | |
| (175) (39) | Severe | | | | | | reduced | |
| Diagnosed | anorectal | NR | 1 | 1 | 2 | 1 | considerably | 3% |
| by | pain, | | | | | | with | |
| colonoscopy/ | bleeding | | | | | | improvement | |
| biopsy | during | | | | | | in | |
| | fecal | | | | | | cachexia. | |
| | elimination, | | | | | | | |
| | cachexia, | | | | | | | |
| | loss of | | | | | | | |
| | appetite. | | | | | | | |
| Rectum | Acute | R | 209 | 210 | 175 | 184 | Improved | 65% |
| (Age 34-68) | pain, | | | | | | cachexia, | |
| M F | bleeding | | | | | | pain no | |
| (165) (97) | during | | | | | | further | |
| Diagnosed | fecal | NR | 2 | 1 | 2 | 3 | problem in | 4% |
| by biopsy | elimination, | | | | | | fecal | |
| | loss | | | | | | elimination, | |
| | of | | | | | | were | |
| | appetite, | | | | | | considerably | |
| | constipating | | | | | | improved in | |
| | and | | | | | | cachexia, | |
| | cachexia | | | | | | no bleeding | |
| | in some | | | | | | during | |
| | cases. | | | | | | fecal | |
| | | | | | | | elimination. | |
| ALL | Severe | R | — | 100 | 130 | — | 70% of the | 70% |
| (Age 4-70) | hepatomegaly, | | | | | | patients | |
| M F | spleenomegaly | | | | | | had a sharp | |
| (105) (45) | persistant | | | | | | improvement | |
| Diagnosed | low | NR | (ND) | 2 | 1 | (ND) | in Hb | 1% |
| by blood | fever, | | | | | | content. | |
| picture and | lack of | | | | | | Normalization | |
| bone marrow | appetite, | | | | | | on WBC | |
| test | cachexia. | | | | | | count and | |
| | | | | | | | platelets. | |
| | | | | | | | In 42 | |
| | | | | | | | patients, | |
| | | | | | | | the bone | |
| | | | | | | | marrow test | |
| | | | | | | | was normal. | |
| AML | Severe | R | — | 40 | 70 | — | Improved | 67% |
| (Age 5-75) | hepatospleenomegally, | | | | | | nausea and | |
| M F | fever, | | | | | | appetite, | |
| (45) (35) | cachexia, | | | | | | normalized | |
| Diagnosed | loss of | NR | (ND) | 1 | 1 | (ND) | hepatospleenomegally, | 1% |
| by bone | appetite | | | | | | reduced | |
| marrow | and | | | | | | body ache | |
| tests and | severe | | | | | | and fever. | |
| blood | body | | | | | | | |
| picture | ache. | | | | | | | |
| Multiple | Cachexia, | R | — | 40 | — | — | Improvement | 72% |
| Myeloma | loss of | | | | | | in general | |
| (Age 30-60) | appetite, | | | | | | conditions, | |
| M F | abnormal | | | | | | increase of | |
| (35) (15) | blood | | | | | | sensory | |
| Diagnosed | picture, | NR | (ND) | 1 | (ND) | (ND) | response. | 1% |
| by biopsy | decrease | | | | | | Bence Jones | |
| and blood | of | | | | | | protein | |
| electrophoresis | sensory | | | | | | negative, | |
| | response | | | | | | A2 | |
| | | | | | | | macroglobulin | |
| | | | | | | | within | |
| | | | | | | | normal | |
| | | | | | | | limits. | |
| Non | Cachexia, | R | — | 240 | 189 | 115 | Reduction | 68% |
| Hodgkin's | acute | | | | | | in the size | |
| Lymphoma | pain, | | | | | | of lymphoma | |
| (Age 24-58) | regional | | | | | | and | |
| M F | lymph | | | | | | metastasis | |
| (158) (100) | adenopathy, | | | | | | of lymph | |
| Diagnosed | loss of | NR | (ND) | 1 | 2 | 4 | node. In | 3% |
| by | appetite, | | | | | | some cases | |
| biopsy/FNAC | huge | | | | | | the swollen | |
| | lymphoma | | | | | | lymph nodes | |
| | in | | | | | | totally | |
| | certain | | | | | | decreased. | |
| | cases. | | | | | | Improvement | |
| | | | | | | | of blood | |
| | | | | | | | picture and | |
| | | | | | | | LFT. | |
| Hodgkin's | Swollen | R | — | 136 | 120 | 71 | Improved | 62% |
| Lymphoma | lymph | | | | | | blood | |
| (Age 17-52) | node | | | | | | picture, | |
| M F | fever, | | | | | | LFT, no | |
| (98) (67) | severe | | | | | | regional | |
| Diagnosed | weakness, | NR | (ND) | 1 | 1 | 3 | lymphadenopathy | 2% |
| by | loss of | | | | | | was | |
| biopsy/FNAC | appetite. | | | | | | evidenced | |
| | | | | | | | by scan and | |
| | | | | | | | FNAC | |
| | | | | | | | reduction | |
| | | | | | | | in swelling | |
| | | | | | | | of nodes | |
| | | | | | | | fever | |
| | | | | | | | normalized. | |
| Uterus | Post menopausal | R | 760 | 800 | 600 | 600 | Improved | 68% |
| (Age 35-60) | bleeding P/R with | | | | | | Hb, WBC | |
| F | acute pain, cachexia. | | | | | | count, LFT, | |
| (1020) | | | | | | | bleeding | |
| Diagnosed | | NR | 5 | 6 | 8 | 6 | and pain | 8% |
| by biopsy, | | | | | | | reduced. | |
| FNAC, USG | | | | | | | Improved | |
| | | | | | | | appetite. | |
| Cervix | Bleeding | R | 875 | 960 | 625 | 510 | Hb, WBC | 67% |
| (age 35-60) | P/R with | | | | | | counts and | |
| F | severe | | | | | | LFT | |
| (998) | pain and | | | | | | normalized, | |
| Diagnosed | cachexia | NR | 3 | 1 | 7 | 9 | reduced | 9% |
| by biopsy, | | | | | | | bleeding | |
| FNAC, USG | | | | | | | and | |
| | | | | | | | abdominal | |
| | | | | | | | distention. | |
| Renal Cell | Acute pain, | R | 160 | 160 | 140 | 110 | Improved | 69% |
| Carcinoma | frequent | | | | | | blood | |
| (Age 35-70) | micturation, | | | | | | picture, | |
| M F | oedema, haematuria, | | | | | | LFT, | |
| (135) (40) | loss of appetite. | | | | | | haematuria | |
| Diagnosed | | NR | 1 | 1 | 1 | 2 | decreased | 2% |
| by | | | | | | | oedema, | |
| FNAC/Cystoscopy/ | | | | | | | electrolytic | |
| biopsy/USG | | | | | | | balance | |
| | | | | | | | restored, | |
| | | | | | | | micturition | |
| | | | | | | | rate | |
| | | | | | | | reduced. In | |
| | | | | | | | some cases | |
| | | | | | | | of bone and | |
| | | | | | | | prostate | |
| | | | | | | | metastasis | |
| | | | | | | | serum PSA | |
| | | | | | | | level | |
| | | | | | | | reduced. | |
| Ovary | Severe | R | 800 | 620 | 600 | 450 | Reduced | 68% |
| (Age 25-60) | bleeding, | | | | | | abdominal | |
| F | white | | | | | | distention, | |
| (900) | discharge, | | | | | | bleeding | |
| Diagnosed | distention | NR | 3 | 8 | 9 | 4 | post- | 8% |
| by biopsy/ | of | | | | | | menopausal | |
| FNAC/USG | lower | | | | | | bleeding, | |
| | abdomen | | | | | | white | |
| | | | | | | | discharge, | |
| | | | | | | | CA-125 | |
| | | | | | | | values | |
| | | | | | | | normalized. | |
| Prostate | Severe pain, | R | 65 | 56 | 67 | 52 | Stabilized | 68% |
| (Age 35-75) | haematuria, frequent | | | | | | Hb levels | |
| M | micturation, oedema of | | | | | | and | |
| (95) | legs, some of them | | | | | | improved | |
| Diagnosed | have bone metastasis, | NR | 1 | 1 | 1 | 1 | LFT, serum | 1% |
| by serum | some were fixed | | | | | | PSA, | |
| PSA, acid | with a catheter. | | | | | | cachexia, | |
| phosphatase | | | | | | | reduced | |
| test, USG | | | | | | | haematuria, | |
| and biopsy | | | | | | | oedema. | |
| | | | | | | | Catheter | |
| | | | | | | | could be | |
| | | | | | | | removed, | |
| | | | | | | | electrolytic | |
| | | | | | | | balance | |
| | | | | | | | could be | |
| | | | | | | | attained. | |
| Glioma | Loss of | R | 80 | 75 | 79 | — | Improvement | 50-60% |
| (Age 30-72) | vision | | | | | | of pain, | |
| M F | paraplegia, | | | | | | appetite, | |
| (65) (37) | cachexia, | | | | | | TC, DC | |
| Evidenced | loss of | NR | 1 | 1 | 1 | (ND) | levels, | 1-2% |
| by CT Scan | memory. | | | | | | tumor | |
| | | | | | | | regression | |
| | | | | | | | (evidenced | |
| | | | | | | | by CT Scan) | |
| | | | | | | | paraplegia, | |
| | | | | | | | neurological | |
| | | | | | | | problems, | |
| | | | | | | | vision, | |
| | | | | | | | memory, | |
| | | | | | | | improved, | |
| | | | | | | | improvement | |
| | | | | | | | in | |
| | | | | | | | generalized | |
| | | | | | | | cachexia. | |
| Tongue | Severe | R | 189 | 178 | 157 | 132 | Improved | 58% |
| (Age 30-65) | pain, | | | | | | hematocrit, | |
| M F | unable to | | | | | | LFT, relief | |
| (168) (57) | speak, | | | | | | from | |
| Diagnosed | eat, | NR | 1 | 1 | 1 | 1 | excrutiating | 2% |
| by punch | excess | | | | | | pain | |
| biopsy, | saliva, | | | | | | in tongue | |
| FNAC | foul | | | | | | and throat. | |
| | odor, | | | | | | Patients | |
| | open | | | | | | markedly | |
| | wounds on | | | | | | improved | |
| | tongue, | | | | | | their | |
| | can only | | | | | | general | |
| | have | | | | | | weakness. | |
| | liquid/ | | | | | | Foul odor | |
| | semisolid | | | | | | and | |
| | diet, | | | | | | constant | |
| | most | | | | | | salivation | |
| | patients | | | | | | reduced. | |
| | reported | | | | | | Subsequent | |
| | severe | | | | | | biopsy | |
| | metastatis | | | | | | revealed | |
| | to | | | | | | absence of | |
| | mandible | | | | | | malignant | |
| | buccal | | | | | | cells. | |
| | mucosa, | | | | | | | |
| | larynx | | | | | | | |
| | and nasopharynx. | | | | | | | |
| Pyriform | Both | R | 145 | 156 | 138 | 146 | Hb, LFT | 49% |
| Fossa | right and | | | | | | improved, | |
| (Age 29-70) | left, | | | | | | ALP, SGOT, | |
| M F | severe | | | | | | SGPT values | |
| (139) (39) | pain, | | | | | | reduced. | |
| Diagnosed | excess | NR | 1 | 1 | 1 | 3 | Improvement | 2% |
| by | saliva, | | | | | | in general | |
| biopsy/FNAC | constipation. | | | | | | weakness, | |
| | | | | | | | voice | |
| | | | | | | | normalized, | |
| | | | | | | | pain | |
| | | | | | | | reduced, | |
| | | | | | | | pain | |
| | | | | | | | killers | |
| | | | | | | | discontinued. | |
| Mandible | Severe | R | 62 | 56 | 49 | 54 | In cases of | 51% |
| (Age 30-65) | pain, | | | | | | hemimandibulectomy | |
| M F | cachexia, | | | | | | and | |
| (42) (33) | metastasis | | | | | | progressive | |
| | to | NR | 1 | 1 | 1 | 1 | metastasis | 1% |
| | nasopharynx, | | | | | | there was | |
| | occipital | | | | | | no further | |
| | region of | | | | | | spread as | |
| | brain | | | | | | evidenced | |
| | some have | | | | | | by CT Scan, | |
| | gone | | | | | | weakness | |
| | through | | | | | | reduced of | |
| | hamimandibulectomy. | | | | | | voice | |
| | | | | | | | improved. | |

Administration of agents of the instant invention was also found to reverse kidney failure Thirty patients with kidney failure and on dialysis (having elevated creatinine levels in the range of 16-17 mg/dL depending on the severity of the disease) were treated with insulin or Na₂Fe[(CN)5NO] in water for approximately 3-6 months depending on the extent of kidney damage. After treatment, there was a 40-60% improvement in kidney function, i.e., creatinine levels decreased to 11-14 mg/dL. Serum creatinine reflects the glomerular filtration rate. Creatinine is a product of creatine metabolism in the muscles, filtered by the kidney but not reabsorbed in the renal tube. A normal creatinine level usually indicates normal kidney function. A rise in creatinine level to three times the normal creatinine suggests 75% loss of renal function. The function of the kidney is to filter the blood through nephrons, selectively reabsorb substances that are needed to maintain the constancy of body fluid and excrete metabolic waste. In end stage renal disease due to diabetes or any other cause there is deterioration of (glomerular) filtration and reduction in functional nephrons. Kidney size is reduced and, among other alterations, fibrous masses form in the capillaries, blood is not filtered properly and normal dialysis is lacking. For survival, treatment is required. Treatment with agents of the instant invention increase nitric oxide levels in the nephrons so that fibrin is dissolved (nitric oxide converts fibrinogen to fibrin) thereby removing obstructions so that filtration can resume, as indicated by a decrease in creatinine levels. Accordingly, agents of the instant invention not only prevent kidney damage but also reduce elevated serum creatinine levels. This type of conservative treatment can retard deterioration of kidney function and assist the body in managing the effects of impaired function. By administering to a patient in need of treatment (e.g., individuals with kidney disease, diabetes, or diseases such as inborn errors in urea cycle enzymes) an effective amount of an agent of the instant invention, creatinine levels are reduced, fibrin is dissolved and kidney function is restored thereby treating kidney failure.

The present invention is further illustrated by the following non-limiting examples.

### EXAMPLE 1

### Patient Selection

A total of 8,125 patients with different kinds of cancer participated in this study. These patients were divided into two groups. Blood samples were collected from 80 patients designated as Group I. These patients included 45 males between 20-65 years old, and 35 females between 25-55 years old. These patients were newly diagnosed patients with cancer who at the time of blood donation had not taken any medicine at least for 14 days and had not yet undergone any treatment of cancer including radiation or chemotherapy but opted for surgery. None of the patients had overt diabetes mellitus, systemic hypertension or suffered coronary artery disease at presentation. None of these patients had any other diagnosed life threatening condition.

Two categories of patients were included in Group II. The first category included 6,705 patients who had previously undergone all available cancer treatments including surgery, radiation and chemotherapy. At the time of participation in the study these patients had exhausted all therapeutic options and were under the care of private physicians and family members in their home. The second category of 1,340 cancer patients in Group II were the patients who for their economic and/or personal reasons did not undergo any conventional treatment for cancer. At the time of the participation in the study all patients, in the Group II in the second category had stopped taking treatments for cancer, such as chemotherapy and radiation, for at least 2 months. The diagnostic procedures and condition of the patients at presentation are described in Table 1. All institutionalized patients in group II were excluded from the study to avoid ambiguity. As in the case of Group I the patients with any other life threatening conditions or severe infection were also excluded from this group.

A control group of 150 patients were selected randomly from the Group II patients. These patients received placebo dermal patches only instead of dermal patches containing agents of the invention. Since the effect of the agents of the instant invention on the neutralization of IANOS antibody in vivo in cancer patients (responders) could be noticed by immunoblot technique (end-point) in 7 days, the patients in the placebo group received the vehicle for 7 days; the neutralization of the antibody and nitric oxide synthesis were determined. After 7 days these patients began to receive treatment with the agents of the invention. In this way responder patients were not denied of any beneficial effects of treatment.

### EXAMPLE 2

### Agent Identification

Both biologic and non-biologic materials for were screened for their ability to activate human erythrocytes IANOS. The biologic material was found to be a bovine pancreatic protein. The protein was purified to homogeneity from an aqueous extract (pH 7.4 buffer) by the combination of DEAE cellulose chromatography and SEPHADEX® gel filtration techniques. The purified protein exhibited an Mr of about 5 kD in alkaline SDS-Polyacrylamide gel electrophoresis. This purified protein identified as insulin and shown to be a potent activator of erythrocyte IANOS.

Insulin for therapeutic use as exemplified herein was prepared by dissolving 0.1-0.2 mg of the protein in 100 mL of 0.9% NaCl containing 0.1% bovine serum albumin with 4% vol/vol glycerol and adjusted to pH 6.8. Any commercial preparation of insulin or other insulin prepared in the laboratory using bovine pancreas could be substituted.

A second agent, identified as sodium nitroprusside was prepared for therapeutic used as exemplified herein by dissolving 10-20 mg Na₂Fe[(CN)5NO] in water and adjusting the solution to pH 6.8.

### EXAMPLE 3

### Administration of Agents

Typically, about 0.2 mL of solution of either of the above identified agents was applied on the absorbent pad in an adhesive bandage. The adhesive bandage was applied to previously cleaned skin, on the lower abdomen, free of hairs, so that the solution soaked pad would tightly adhere to the skin. If required, the patch was replaced by a new one every 24 hours. Although the patch of agent thus prepared was usually applied on the lower abdominal area, any other suitable part of the body can be used. In in vivo experiments with animals, the patch was similarly applied on the skin except that before application the hairs on the abdomen area of the animal were shaved and the skin was cleaned.

Collection of blood and preparation of plasma and erythrocyte membrane blood was collected from normal healthy volunteers or from Group I cancer patients. The normal volunteers (n=50) had not taken any medication for at least for 14 days prior to the donation of blood. Only age- and sex-matched volunteers participated in the study.

### EXAMPLE 4

### Assay of IANOS of Human Erythrocytes

Insulin-activated nitric oxide synthase (IANOS) activity of the erythrocyte suspension was carried out by determining the conversion of oxyhemoglobin to methemoglobin using standard methods. Purification of insulin activated nitric oxide synthase (from human erythrocyte membranes) was carried out by DEAE cellulose chromatography.

### EXAMPLE 5

### Characterization of the Plasma IANOS Inhibitor

Purified inhibitor was immunoblotted with ¹²⁵I-labeled anti-human, anti-IgG, which in turn was conjugated to protein-A. The immunoblot was performed and quantified.

### EXAMPLE 6

### Statistical Analysis

Because of the large number of patients (8,045) with different types of cancer in each cancer category and multi-variant parameters, the significance of the effect of the agents of the instant invention in responders was analyzed by tau test (student “t” test tends to tau large number of ‘n’). The acceptance of rejection of the significance in all 26 types of cancer tested by null hypothesis of the 5% level of significance indicated 95% level of acceptance.

1. A method for preventing and treating cancer without causing significant side effects comprising topically administering to a human patient in need of prevention or treatment of cancer an agent which modulates the production of nitric oxide by cells found in the epidermal tissue layer of the patient thereby preventing and treating cancer without causing significant side effects.

2. The method of claim 1, wherein the agent is topically administered via a dermal patch.

3. The method of claim 1, wherein the agent comprises insulin.

4. The method of claim 1, wherein the agent comprises Na₂Fe[(CN)5NO].

5. The method of claim 1, further comprising administering aspirin.

6. A method for relieving pain without causing significant side effects comprising topically administering to a patient in need of treatment an agent which modulates the production of nitric oxide by red blood cells of the patient thereby relieving pain without causing significant side effects.

7. A method for decreasing the systemic side effects of cancer treatment comprising topically administering to a patient receiving cancer treatment an agent which modulates the production of nitric oxide by cells found in the epidermal tissue layer of the patient thereby decreasing the systemic side effects of the cancer treatment.

8. A method for controlling or preventing diabetes comprising topically administering to a patient with diabetes or at risk of diabetes an agent which modulates the production of nitric oxide by cells found in the epidermal tissue layer of the patient thereby controlling or preventing diabetes.

9. The method of claim 7 wherein the patient is a cancer patient.

10. The method of claim 7 wherein the patient is a type I or type II diabetic.

11. A method for treating cancer and relieving pain without causing significant side effects comprising topically administering to a cancer patient an agent which modulates the production of nitric oxide by red blood cells thereby treating the cancer and relieving pain without causing significant side effects.

12. A method for treating kidney failure comprising topically administering to a human patient in need of treatment an agent which modulates the production of nitric oxide by cells of the patient so that kidney function is restored.
